(19) United States
(12) Patent Application Publication
(10) Pub. No.: US 2014/0207101 A1
(43) Pub. Date: Jul. 24, 2014

(54) MICROSTRUCTURE DEVICE WITH REMOVABLE MICROSTRUCTURE PATCH

(71) Applicant: 3M INNOVATIVE PROPERTIES COMPANY, St. Paul, MN (US)

(72) Inventors: Craig S. Moeckly, White Bear Lake, MN (US); Dennis G. Berry, Maplewood, MN (US); Ryan Patrick Simmers, Cottage Grove, MN (US); Erik J. Johnson, Cohasset, MN (US); Richard D. Smasal, Afton, MN (US)

(21) Appl. No.: 14/118,904

(22) PCT Filed: Jun. 6, 2012
(86) PCT No.: PCT/US2012/041056

§ 371 Date: Nov. 20, 2013

Related U.S. Application Data
(60) Provisional application No. 61/494,944, filed on Jun. 9, 2011.

(51) Int. Cl. A61M 37/00

(52) U.S. Cl. CPC A61M 37/0015
USPC 604/506; 604/173

(57) ABSTRACT

A device and method for creating microchannels in the stratum corneum. The device includes a handle and a substrate detachably attached to the handle by a coupler. The substrate has a plurality of microstructures for creating microchannels in the stratum corneum. The substrate is detachable from the handle for disposal.

### CROSS REFERENCE TO RELATED APPLICATIONS

This application claims the benefit of U.S. Provisional Patent Application No. 61/494,944, filed Jun. 9, 2011, which is incorporated herein by reference in its entirety.

### BACKGROUND

The outermost layer of the epidermis, the stratum corneum, protects the body against many physical and chemical assaults. These layers of dead, flat cells keep hydration in the body and keep out biological, bacterial, and viral invaders. When the stratum corneum is disrupted in a controlled fashion, compounds, such as drugs, that normally cannot penetrate this protective barrier, can be delivered locally or systemically. This disruption can enable delivery of a systemic drug or precise, targeted delivery for localized drug delivery, such as topical anesthetics, anti-inflammatories, or corticosteroids. Such disruption of the stratum corneum prior to drug delivery has been done using mechanisms such as abrasion, laser ablation, and radio-frequency.

### BRIEF SUMMARY

This disclosure relates to a device and method for creating microchannels in the stratum corneum. The device includes a handle and a substrate detachably attached to the handle by a coupler. The substrate has a plurality of microstructures for creating microchannels in the stratum corneum. The substrate is detachable from the handle for disposal.

The features and advantages of this disclosure will be understood upon consideration of the detailed description, as well as the appended claims. These and other features and advantages of the invention may be described below in connection with various embodiments of the invention. The above summary of the present invention is not intended to describe each disclosed embodiment or every implementation of the present invention.

The subject matter of this disclosure, in its various combinations, either in apparatus or method form, may include the following list of embodiments:

1. A microstructure device for creating microchannels in the stratum corneum, said microstructure device comprising:

a handle; and

a substrate detachably attached to said handle by a coupler presented on said substrate, said substrate comprising a face and a plurality of microstructures presented on said face for creating microchannels in the stratum corneum,

wherein said substrate is detachable from said handle for disposal of said substrate by effecting movement of said coupler presented on said substrate.

2. The microstructure device of embodiment 1, wherein said coupler comprises a protrusion operably engaged with said handle to attach said substrate to said handle.

3. The microstructure device of embodiment 2, wherein said handle comprises an aperture, said protrusion engaged with said aperture to attach said substrate to said handle.

4. The microstructure device of embodiment 3, wherein said movement disengages said protrusion from said aperture such to effect detachment of said substrate from said handle.

5. The microstructure device of embodiment 3, wherein said protrusion comprises a recess engaged with said handle to attach said substrate to said handle.

6. The microstructure device of embodiment 5, wherein said movement disengages said protrusion from said handle to effect detachment of said substrate from said handle.

7. The microstructure device of embodiment 5, wherein movement effects flexion of said protrusion, such that said recess disengages from said substrate to effect detachment of said substrate from said handle.

8. The microstructure device of any of the preceding embodiments, wherein the face is generally planar.

9. The microstructure device of any of the preceding embodiments, wherein said microstructures comprise microneedles.

10. The microstructure device of any of the preceding embodiments, wherein said microstructures comprise solid microneedles.

11. The microstructure device of any of the preceding embodiments, wherein said substrate comprises a polymeric material.

12. The microstructure device of any of the preceding embodiments, wherein said substrate comprises a liquid crystal polymer.

13. A microstructure patch for creating microchannels in the stratum corneum, said microstructure patch comprising:

a substrate comprising a face and a plurality of microstructures presented on said face for creating microchannels in the stratum corneum; and

a coupling member presented on said substrate for detachably attaching said substrate to a handle, wherein said substrate is detachable from the handle for disposal of said substrate by effecting movement of said coupling member.

14. The microstructure patch of embodiment 13, wherein the face is generally planar.

15. The microstructure patch of any one of embodiments 13 to 14, wherein said microstructures comprise microneedles.

16. The microstructure patch of any one of embodiments 13 to 15, wherein said microstructures comprise solid microneedles.

17. The microstructure patch of any one of embodiments 13 to 16, wherein said substrate comprises a polymeric material.

18. The microstructure patch of any one of embodiments 13 to 17, wherein said substrate comprises a liquid crystal polymer.

19. A method of using a microstructure patch for creation of microchannels in the stratum corneum, said method comprising:

providing a handle and a substrate detachably attached to said handle by a coupler presented on said substrate, said substrate comprising a face a plurality of microstructures presented on said face for creating microchannels in the stratum corneum; applying said plurality of microstructures to the stratum corneum to created microchannels; and after microchannels are created in the stratum corneum, detaching said substrate from said handle by effecting movement of said coupler presented on said substrate.

20. The method of embodiment 19, wherein said coupler comprises a coupling member and said handle comprises an aperture, said coupling member engaged with said aperture to attach said substrate to said handle, wherein said movement disengages said coupling member from said aperture such that said substrate detaches from said handle.

21. The method of embodiment 20, wherein said coupling member comprises a recess engaged with said handle to attach said substrate to said handle, and wherein said movement disengages said recess from said handle to effect detachment of said substrate from said handle.

22. The method of embodiment 20, wherein said coupling member comprises a recess, said recess engaging at least a portion of said handle to attach said substrate to said handle, and wherein said movement effects flexion of said coupling member, such that said recess disengages from said substrate to detach said substrate from said handle.

23. A kit for creating microchannels in the stratum corneum, said kit:

a microstructure patch presented in a package, each microstructure patch comprising a plurality of microstructures for creating microchannels in the stratum corneum; and

instructions comprising the steps of:

removing said microstructure patch from said package using a handle;

applying said plurality of microstructures to the stratum corneum to create microchannels in the stratum corneum; and

after microchannels are created in the stratum corneum, detaching said microstructure patch from the handle for disposal.

24. The kit of embodiment 23, wherein said microstructure patch further includes a coupling member and said instructions further include the step of effecting movement of said coupling member to detach said microstructure patch from the handle.

25. The kit of embodiment 24, wherein the handle comprises an aperture and said coupling member is presented on said substrate, said coupling member engaged with said aperture to attach said microstructure patch to the handle, wherein said movement disengages said coupling member from the aperture such that said microstructure patch detaches from the handle.

26. The kit of any one of embodiments 24 to 25, wherein said coupling member comprises a recess engaged with said handle, and wherein said movement disengages said recess from said handle to effect detachment of said microstructure patch from the handle.

27. The kit of any one of embodiments 24 to 25, wherein said coupling member comprises a recess engaged with said handle, and wherein said movement effects flexion of said coupling member to effect detachment of said microstructure patch from the handle.

### BRIEF DESCRIPTION OF THE SEVERAL VIEWS OF THE FIGURES

The disclosure can be more completely understood in consideration of the following detailed description in connection with the accompanying figures, in which:

FIG. 1 is a top perspective view of a microstructure device;

FIG. 1A is a cross-sectional view of the microstructure device of FIG. 1;

FIG. 1B is a close-up fragmentary view of a portion of the cross-section of FIG. 1A;

FIG. 2 is a fragmentary view of the microstructure device of FIG. 1;

FIG. 3 is a bottom perspective view of a microstructure patch of the microstructure device of FIG. 1;

FIG. 4 is an elevational view of the microstructure patch of FIG. 3;

FIG. 5 is a top perspective view of a handle of the microstructure device of FIG. 1; and

FIG. 6 depicts a user using a handle of FIG. 5 to pick up the microstructure patch of FIG. 3 from packaging.

While the above-identified figures depict an embodiment of the disclosed subject matter, other embodiments are also contemplated, such as those noted in the disclosure. This disclosure presents the disclosed subject matter by way of representation only and not by limitation. The figures are representations, for which reason the configuration of the different structures, as well as their relative dimensions, is for illustrative purposes only. Numerous modifications and embodiments can be recognized by those skilled in the art, which modifications and embodiments are within the scope and spirit of this disclosure.

### DETAILED DESCRIPTION

A microstructure device according to this disclosure can generally include a handle and a removable patch having an array of microstructures that can be used to disrupt the stratum corneum, in a comfortable, convenient, and controlled fashion, by creating microchannels in the skin. Such disruption of the stratum corneum can be used for various medical and cosmetic applications. As an example, in dermatology applications, the microstructure device can be used for skin rejuvenation to treat conditions, such as wrinkles, acne, and scars. The microstructure device can also be used for wound debridement, disruption of biofilms, and other wound care applications, as well as for pre-treatment of the skin prior to delivery of various medicaments, such as numbing agents (such as lidocaine), steroids, anti-infectives, Botulinum toxin, hyaluronic acid, as well as for pre-treating the skin prior to use of topicals in highly-keratinized tissue, such as atopic dermatitis, actinic keratosis, eczema, and psoriasis.

The microstructure device can further be used for pre-treatment of the skin prior to delivery of various medicaments, such as, for example, Botulinum toxin or vaccines, as well as for pre-treating the skin prior to use of topical medicaments, such as, for example, Vitamin C or hyalurinidases. The microstructure patch portion can be packaged in sterile packaging and can be reused or disposed of after a single treatment session. Likewise, the handle can be reusable or also disposed of after a single treatment session.

In this disclosure, including in the following description, certain terms will be understood to have the meaning set forth below:

“Array” refers to one or more structures capable of piercing the stratum corneum.

“Microstructure” refers to structures associated with the array that are capable of piercing the stratum corneum. By way of example, microstructures can include needle or needle-like structures as well as other structures, such as blades or projections, capable of piercing the stratum corneum.

When in the following terms such as “upper” and “lower”, “top” and “bottom”, “right” and “left”, “first” and “second”, or similar relative expressions are used, these terms only refer to the appended figures and not necessarily to an actual situation of use.

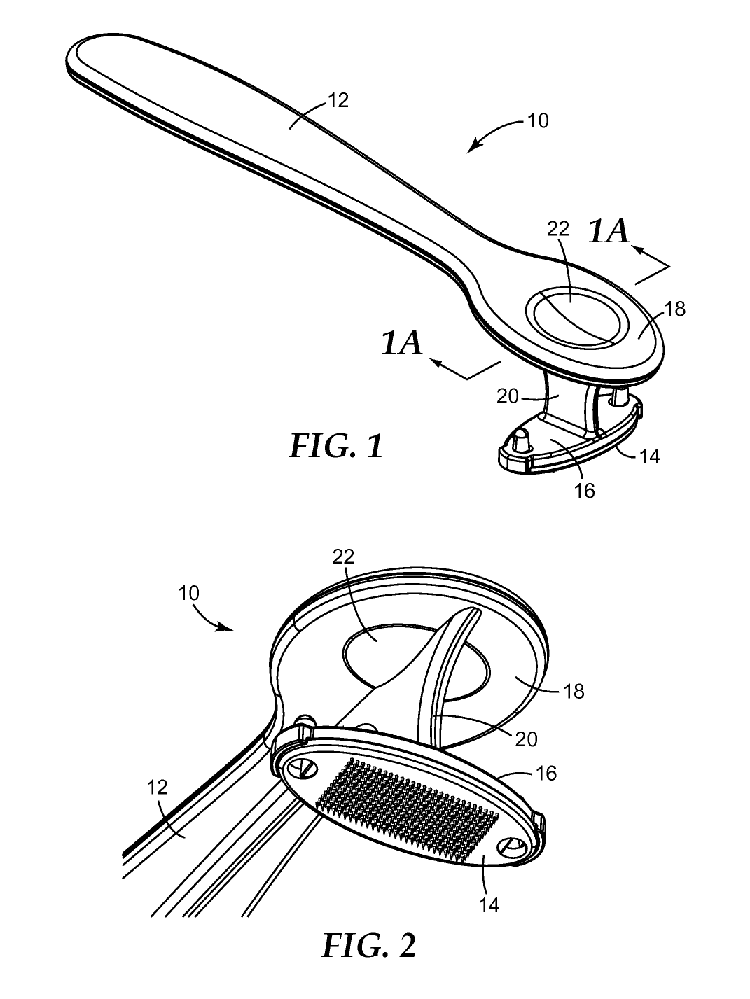

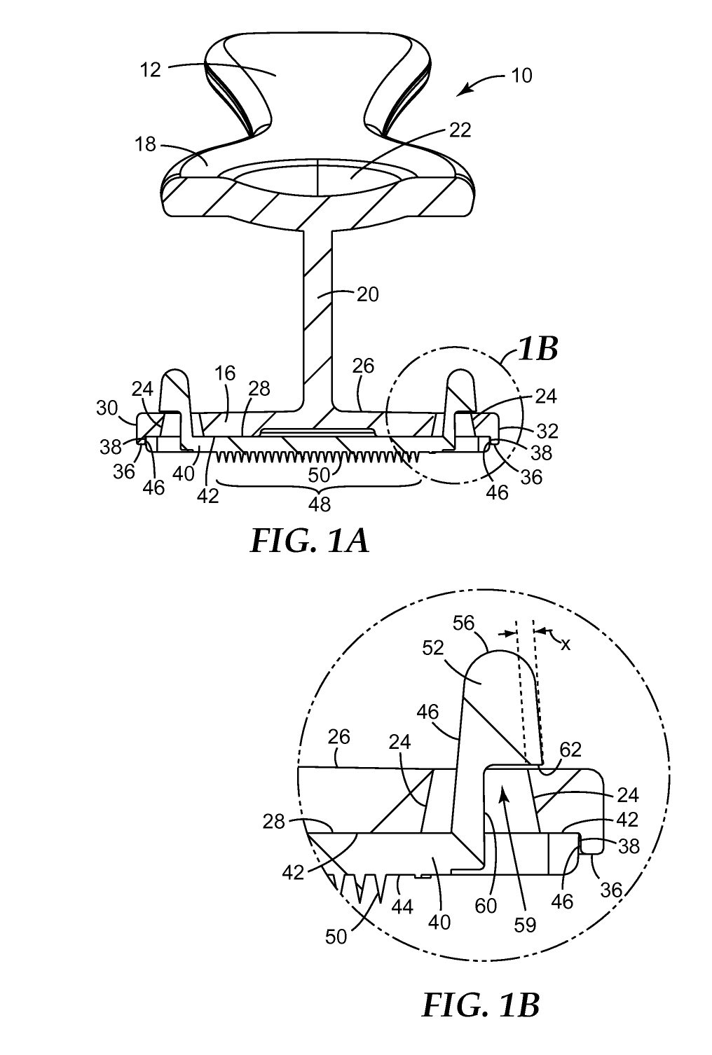

Referring to FIGS. 1 and 2, a microstructure device 10 generally includes a handle 12 and a microstructure patch 14 detachably attached to handle 12. Handle 12 includes an array head 16 extending from handle 12, such as at an end 18 of handle 12. Head 16 can be configured for selectively and removeably attaching microstructure patch 14 to handle 12. As depicted, head 16 is generally oval-shaped or oblong, although those skilled in the art would recognize that head 16 can be a number of shapes, including circular or polygonal shapes. Head 16 can extend from end 18 of handle 12 by a rib 20, which rib 20 can also span partially or fully along an entire length of handle 12 to provide structural integrity and/or aesthetic appeal to microstructure device 10. Rib 20 can also provide enhanced gripping of handle 12 by a user. End 18 of handle 12 can include a depression 22 presented thereon to enable a user to place his or her finger or thumb when grasping and using handle 12 for increased control or leverage. Depression 22 can also provide further aesthetic appeal to microstructure device 10. Handle 12 can comprise the depicted shape to provide handling comfort and/or aesthetic appeal to microstructure device 10, although those skilled in the art would recognize that handle 12 can have a number of alternative shapes (such as, for example, a trowel shape, a razor shape, e.g., a disposable or reusable razor shape, or a stamp shape).

Referring to FIGS. 1A and 1B, head 16 can include one or more apertures 24 extending between a first, upper surface 26 of head 16 and a second, lower surface 28 of head 16. As depicted, head 16 includes two apertures 24, one aperture 24 proximate a first end 30 of head 16 and another aperture 24 presented proximate a second end 32 of head 16. Second surface 28 can be generally parallel to first surface 26. As depicted, apertures 24 can be tapered, inwardly, from second surface 28 towards first surface 26. Referring to FIG. 5, apertures 24 can be generally circular, but can include a generally flat edge or surface 34 therein for mating with a recess 58 on coupling members 52 included on microstructure patch 14—discussed in greater detail below. Head 16 can further include a plurality of flanges or lips 36 extending from second surface 28 of head 16, such as at first end 30 and second end 32 of head 16, each of flanges 36 including an inner surface 38 presented thereon. In embodiments, handle 12 can be formed of ABS, such as LUSTRAN ABS 240 available from Ineous, using injection molding, although those skilled in the art would understand that handle 12 can be formed of other materials, such as, for example, metals, other polymers or co-polymers, or ceramics, and by other processes, such as, for example, casting, machining, lithography, etching, and other molding processes.

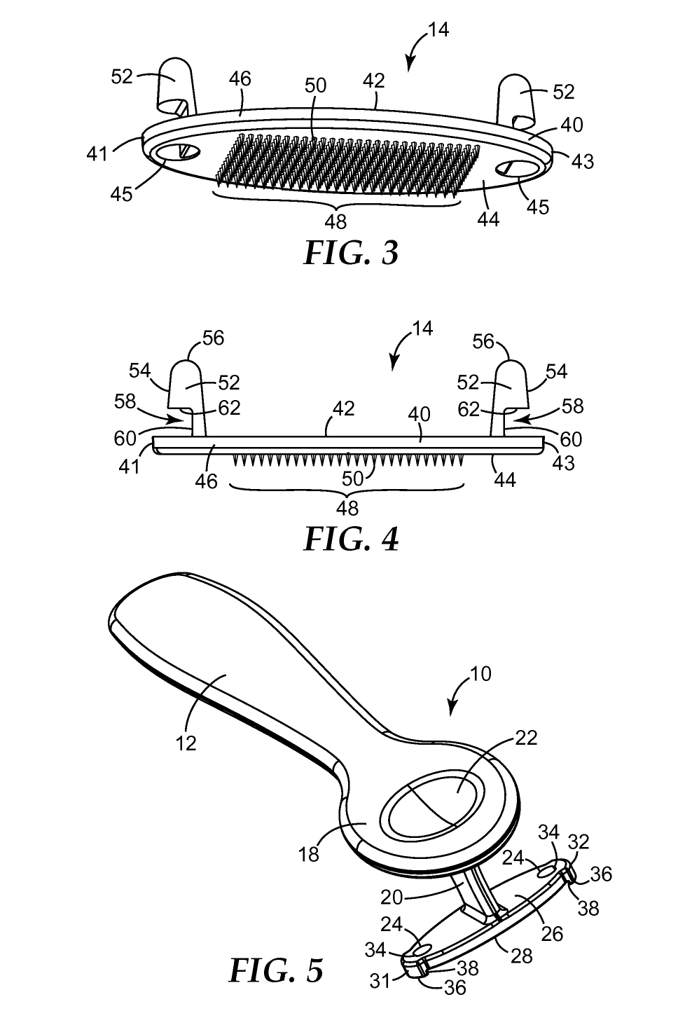

Referring to FIGS. 3 and 4, microstructure patch 14 can include a substrate 40 having a first, upper surface 42, a second, lower surface or face 44 generally opposed and parallel to first surface 42, and an outer surface 46 extending between first surface 42 and face 44. As depicted, microstructure patch 14 is generally oval-shaped or oblong, having a first end 41 and a second end 43, although those skilled in the art would recognize that microstructure patch 14 can be a number of shapes, including circular or polygonal shapes. Microstructure patch 14 further includes a grid or array 48 of microstructures 50 presented on face 44 and one or more coupling members or projections 52 (two coupling members 52 depicted) extending or projecting from first surface 42 (one coupling member 52 being position proximate first end 41 and the other coupling member 52 being position proximate second end 43). The number of coupling members 52 can correspond with the number of apertures 24 included on handle 12. Also, the positioning of coupling members 52 on substrate and the positioning of apertures 24 on handle 12 can be such that coupling members 52 are aligned with apertures 24 when patch 14 or substrate 40 is detachably attached to handle 12 (discussed below).

Microstructures 50 can have various shapes and sizes, such as described in the following patents and patent applications, the disclosures of which are incorporated herein by reference in their entirety, including, without limitation, their disclosure of microstructure configurations: U.S. Patent Application Publication No. 2005/0261631 (Clarke et al.), which describes microneedles having a truncated tapered shape and a controlled aspect ratio; U.S. Pat. No. 6,091,975 (Daddona et al.), which describes blade-like microprotrusions for piercing the skin; U.S. Pat. No. 6,312,612 (Sherman et al.), which describes tapered structures having a hollow central channel; and U.S. Pat. No. 6,379,324 (Gartstein et al.), which describes hollow microneedles having at least one longitudinal blade at the top surface of the tip of the microneedle. Other patents and publications that describe microstructure configurations, the disclosures of which are incorporated herein by reference in their entirety, including, without limitation, their disclosure of microstructure configurations, are as follows: U.S. Pat. No. 6,219,574 (Cormier et al.), U.S. Pat. No. 7,537,795 (Cormier et al.), and Ameri et al., “Parathyroid Hormone PTH(1-34) Formulation that Enables Uniform Coating on a Novel Transdermal Microprojection Delivery System,” Pharmaceutical Research, 2009.

In an embodiment, microstructures 50 have a square-pyramidal shape with a base width of about 0.0111 inches, a spacing of about 0.0200 inches (measured from microstructure peak to peak), and a height of about 0.0276 inches. Array 48 can include any number of microstructures 50. In a particular embodiment, as depicted, array includes 351 microstructures 50 (array of 13 microstructures by 27 microstructures).

Coupling members 52 include an outer surface 54 that can taper inwardly in a direction moving away from substrate 40 and a generally rounded tip 56. Coupling members 52 can further include a recess 58 therein extending a portion of a length of coupling members 52. Recess 58 can include a first portion 62 that can be generally parallel first surface 42 of substrate 40 and a second portion 60 that can be generally transverse first portion 62 and first surface 42 of substrate 40. Microstructure patch 14 can be formed using the processes described in PCT Publication No. WO2006/062974, entitled “Method of Molding a Microneedle,” PCT Publication No. WO2007/112309, entitled “Process for Making Microneedles, Microneedle Arrays, Masters, and Replication Tools,” PCT Application No. PCT/US2011/038026, entitled “Liquid Crystalline Polymer Microneedles,” and PCT Publication No. WO2011/075569, entitled “Molding Thermotropic Liquid Crystalline Polymers and Articles Made Therefrom,” each of which are incorporated herein by reference in their entirety, including, without limitation, their disclosures of methods of forming microstructure patches. In an embodiment, microstructure patch 14 can be injection molded from Class VI, Medical Grade liquid crystalline polymer (LCP), such as VECTRA MT1300, available from Ticona Plastics, Auburn Hills, Mich., although those skilled in the art would understand that microstructure patch 14 and microstructures 50 can be formed of (or include) other materials, such as silicon or a metal, such as stainless steel, titanium, or nickel titanium alloy. The microneedle material can also be (or include) other medical grade polymeric materials. Exemplary types of other medical grade polymeric materials include, for example, polycarbonate.

Those skilled in the art will recognize that while recesses 58 of coupling members 52 are depicted as being generally inwardly oriented relative to outer surface 46 of substrate, recesses 58 can be outwardly oriented or oriented in other directions relative to outer surface 46 of substrate. Also, while recesses 58 of coupling members 52 are depicted as being oriented in opposite directions, i.e., one recess 58 is oriented in one direction and the other recess 58 is oriented in the opposite direction, each of recesses 58 can be oriented in the same direction or oriented in directions that generally does not correspond to the orientation of the recess 58 presented in the other coupling member 52.

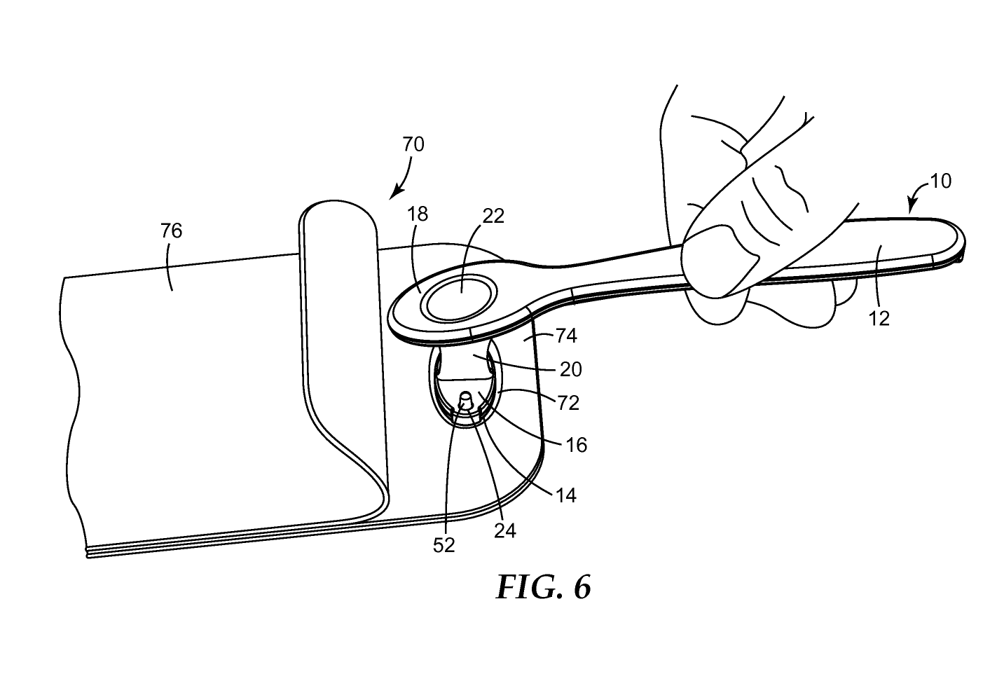

Referring to FIG. 6, which depicts use of handle 12 to collect a microstructure patch 14 contained within packaging 70, which, optionally, can be sterile packaging, such as a blister pack. Packaging 70 can include one or more cavities 72 formed in a web 74 made of, for example, a thermoformed plastic. Packaging 70 can further include a lid or sealing member 76 that can be made of, for example, aluminum foil or plastic. A microstructure patch 14 can be positioned in each of cavities 72 and then sealed in with lid or sealing member 76. Such microstructure patch 14 containers can be used to inhibit handling the microstructure patch 14 prior to use. Also, by including the removable and disposable microstructure patch 14 in packaging 70, but not handle 12, which can be reusable, waste material can be minimized.

In use, a user, such as a physician or an individual, peels back lid 76 on packaging 70, which exposes cavity 72 and first surface 42 of microstructure patch 14 and coupling members 52 presented in cavity 72. Coupling members 52 on microstructure patch 14 can then be aligned with apertures 24 in head 16 of handle 12 and a user can press gently downwardly into cavity 72 so that coupling members 52 move or slide into apertures 24. As a user does this, rounded tip 56 of coupling members 52, and then outer surfaces 54, which can be tapered, will slide along apertures 24, which can be tapered, and coupling members 52 can flex inwardly. This movement and flexion will continue until first portions 62 of recesses 58 have traveled past upper surfaces 26 of head 16, so that coupling members 52 return to an unflexed state and second portions 62 of recesses 48 are adjacently positioned flat edges 34 of apertures 34—snapping coupling members 52 into place. Referring to FIG. 1B, in this configuration, the overlap or interference “x” between first portion 62 of recess 58 and upper surface 26 of head 16 is greater than zero, yet minimized so that coupling members can easily disengage for later disposal of microstructure patch 14, discussed below. “x” can be less than about 0.0250 inches. Preferably, x is less than about 0.0170 inches. Optimally, x is less than about 0.0115 inches.

After microstructure patch 14 has been attached to head 16 of handle 12, array 48 of microstructures 50 can be applied to and pressed against the stratum corneum for a period of time, which creates microchannels in skin. Preferably, the application is done in a straight up and down motion in a direction transverse to the skin. The application of microstructures 50 is then removed and, if desired, topical product is applied to the pre-treated area.

Following treatment with microstructure patch 14, microstructure patch 14 can be ejected or discharged from head 16 of handle 12 by pressing in or down on one or both of coupling members 52. Such pressing can cause inward movement or flexion of the one or more coupling members 52, such that coupling members 52 flex inwardly at least a distance greater than or equal to “x.” When one of coupling members 52 is pressed, flange 36 on the opposite end of head 16 from the coupling member 52 being pressed will retain the patch on head during this process and inhibit or prevent movement of patch 14 during such pressing.

To further describe the mechanism enabling ejection of microstructure patch 14 following treatment, when coupling member 52 is pressed causing inward movement or flexion of the coupling member 52, first portion 62 of recess 58 will clear (or pass beyond) upper surface of head 16 and coupling member 52 will then travel along apertures 24 in a direction back down and beyond past upper surface 26 of head 16. Once first portion 62 of recess 58 has passed beyond upper surface of head 16, coupling member 52 will again be flexed inwardly, this time causing the outer surface 54, and then the rounded tip 56, to slide along apertures 24 until tip 56 is beyond bottom surface 28 of head 16. Once this occurs, coupling member 52 will be in its unflexed state and the other of the coupling members 52 will disengage with its respective aperture 24—such that the microstructure patch 14 is ejected or discharged from handle 12. Such ejection enables microstructure patch 14 to be disposed of easily, without it being “hung up” on head 16 of device 10 and without any need for a user to handle the patch other than the initial pressing of one or more of coupling members 52.

Although the microstructure device and methods disclosed herein has been described with respect to various embodiments, those skilled in the art will recognize that changes may be made in form and detail without departing from the spirit and scope of the microstructure device and methods disclosure. As an example, while coupling members or couplers 52 have been depicted and described as being presented on microstructure patch 14, such coupling members or couplers 52 can be alternatively (or additionally) presented on handle 12.

1. A microstructure device for creating microchannels in the stratum corneum, said microstructure device comprising:
a handle; and
a substrate detachably attached to said handle by a coupler presented on said substrate, said substrate comprising a face and a plurality of microstructures presented on said face for creating microchannels in the stratum corneum,
wherein said substrate is detachable from said handle for disposal of said substrate by effecting movement of said coupler presented on said substrate.

2. The microstructure device of claim 1, wherein said coupler comprises a protrusion operably engaged with said handle to attach said substrate to said handle.

3. The microstructure device of claim 2, wherein said handle comprises an aperture, said protrusion engaged with said aperture to attach said substrate to said handle.

4. The microstructure device of claim 3, wherein said movement disengages said protrusion from said aperture such to effect detachment of said substrate from said handle.

5. The microstructure device of claim 3, wherein said protrusion comprises a recess engaged with said handle to attach said substrate to said handle.

6. (canceled)

7. The microstructure device of claim 5, wherein movement effects flexion of said protrusion, such that said recess disengages from said substrate to effect detachment of said substrate from said handle.

8. The microstructure device of claim 1, wherein the face is generally planar.

9. The microstructure device of claim 1, wherein said microstructures comprise microneedles.

10. The microstructure device of claim 1, wherein said microstructures comprise solid microneedles.

11. The microstructure device of claim 1, wherein said substrate comprises a polymeric material.

12. The microstructure device of claim 1, wherein said substrate comprises a liquid crystal polymer.

13. A microstructure patch for creating microchannels in the stratum corneum, said microstructure patch comprising:
a substrate comprising a face and a plurality of microstructures presented on said face for creating microchannels in the stratum corneum; and
a coupling member presented on said substrate for detachably attaching said substrate to a handle, wherein said substrate is detachable from the handle for disposal of said substrate by effecting movement of said coupling member.

14. The microstructure patch of claim 13, wherein the face is generally planar.

15. The microstructure patch of claim 13, wherein said microstructures comprise microneedles.

16. The microstructure patch of claim 13, wherein said microstructures comprise solid microneedles.

17. The microstructure patch of claim 13, wherein said substrate comprises a polymeric material.

18. The microstructure patch of claim 13, wherein said substrate comprises a liquid crystal polymer.

19. A method of using a microstructure patch for creation of microchannels in the stratum corneum, said method comprising:
providing a handle and a substrate detachably attached to said handle by a coupler presented on said substrate, said substrate comprising a face a plurality of microstructures presented on said face for creating microchannels in the stratum corneum;
applying said plurality of microstructures to the stratum corneum to created microchannels; and
after microchannels are created in the stratum corneum, detaching said substrate from said handle by effecting movement of said coupler presented on said substrate.

20. The method of claim 19, wherein said coupler comprises a coupling member and said handle comprises an aperture, said coupling member engaged with said aperture to attach said substrate to said handle, wherein said movement disengages said coupling member from said aperture such that said substrate detaches from said handle.

21. The method of claim 20, wherein said coupling member comprises a recess engaged with said handle to attach said substrate to said handle, and wherein said movement disengages said recess from said handle to effect detachment of said substrate from said handle.

22. The method of claim 20, wherein said coupling member comprises a recess, said recess engaging at least a portion of said handle to attach said substrate to said handle, and wherein said movement effects flexion of said coupling member, such that said recess disengages from said substrate to detach said substrate from said handle.

23. A kit for creating microchannels in the stratum corneum, said kit:
a microstructure patch presented in a package, each microstructure patch comprising a plurality of microstructures for creating microchannels in the stratum corneum; and
instructions comprising the steps of:
removing said microstructure patch from said package using a handle;
applying said plurality of microstructures to the stratum corneum to create microchannels in the stratum corneum; and
after microchannels are created in the stratum corneum, detaching said microstructure patch from the handle for disposal.

24. The kit of claim 23, wherein said microstructure patch further includes a coupling member and said instructions further include the step of effecting movement of said coupling member to detach said microstructure patch from the handle.

25. The kit of claim 24, wherein the handle comprises an aperture and said coupling member is presented on said substrate, said coupling member engaged with said aperture to attach said microstructure patch to the handle, wherein said movement disengages said coupling member from the aperture such that said microstructure patch detaches from the handle.

26-27. (canceled)
